(19) United States
(12) Patent Application Publication
(10) Pub. No.: US 2019/0218588 A1
(43) Pub. Date: Jul. 18, 2019

(54) NOVEL CELL CULTURE METHOD, CELL CULTURE SYSTEM AND USES THEREOF

(71) Applicant: Albert LI, Columbia, MD (US)

(72) Inventor: Albert LI, Columbia, MD (US)

(21) Appl. No.: 16/303,422

(22) PCT Filed: May 23, 2017
(86) PCT No.: PCT/US17/34048

§ 371 Date: Nov. 20, 2018

Related U.S. Application Data
(60) Provisional application No. 62/340,702, filed on May 24, 2016.

(51) Int. Cl. C12Q 1/02; C12N 5/071

(52) U.S. Cl. CPC C12Q 1/025; C12N 5/067; G01N 2800/085; G01N 2500/10; C12N 2502/11; C12N 2533/54; C12N 2500/84

(57) ABSTRACT

A method is disclosed wherein human organ cells (e.g. hepatocytes) are cultured in human plasma medium (e.g., 70% to 100% human plasma) in place of the routinely used non-human plasma cell culture growth medium. This culture method allows the human organ cells to be in an environment that closely resembles that of the in vivo environment, where the cells are bathed in human plasma. Also, disclosed herein are cell culture systems containing the human organ cells and human plasma medium in a cell culture vessel. Uses of the cell culture system includes application of cultured human organ cells to evaluate test compound properties, including pharmacological, pharmacokinetic, and toxicological effects of drugs, organ disease progression, such as liver disease progression including hepatitis B infection or hepatitis C infection, or cell biology process, such as gene expression, protein synthesis or response to hormones, that can be directly translated to human in vivo.

### CROSS-REFERENCE TO RELATED APPLICATIONS

This application is a 371 application, US National Phase Application, of International Application No. PCT/2017/034048, filed 23 May 2017, which claims the benefit of U.S. Provisional Patent Application No. 62/340,702, filed on 24 May 2016, the contents of which are incorporated herein by reference in their entirety.

### FIELD OF THE INVENTION

The disclosed inventions relate generally to cell culture methods, cell culture systems and methods using the cultured cells for the evaluation of drug pharmacological activities, drug metabolism, drug-drug interactions, and drug toxicity.

### BACKGROUND OF THE INVENTION

The invention relates to cell culture methods of organ cells (e.g. hepatocytes) in human plasma, and use of those cultured cells for the evaluation of drug pharmacological activities, drug metabolism, drug-drug interactions, and drug toxicity.

In vitro cell culture experimental systems are routinely used for biomedical research. A major difference between cell culture and a mammalian organism in vivo is that cell culture is performed in culture medium containing artificial levels of nutrients and often supplemented with animal products, such as, for example, fetal calf serum.

Until now, cell culture media for human cells have been chemically designed to maintain viability and proliferation. Improvements in chemically defined cell culture medium have been provided to support clonal growth of hepatic cell lines, genetically transformed hepatocytes, and hepatocytes obtained from neoplastic sources. Those complex culture environments may include hepatocyte cell growth promoting substances such as, for example, nicotinamide, amino acids, transferrin, hormones, dexamethasone, trace metals, carbohydrates, buffering agents, and albumin, some or all of which may be exogenous to the cell being cultured. A major challenge is the culturing of primary cells—cells derived from normal tissues to retain functions as that observed for the cells in vivo.

In contrast to in vitro cell culture, in an animal or a human in vivo, cells in each organ are nourished by circulating blood which consists of endogenous plasma and blood cells. The difference in environment makes it difficult for observations made using cell culture system to be directly extrapolated to events in vivo.

An important cell culture system is primary cultured human hepatocytes which are generally considered as the “gold standard” for in vitro evaluation of drug properties such as drug metabolism, drug-drug interaction, and pharmacological effects. A major challenge is the translation of events observed in hepatocyte cultures to that in vivo as the properties of human hepatocytes maintained in artificial culture medium are known to be different from the liver in vivo. An additional compounding factor is the binding of the drugs being investigated to plasma proteins.

The invention disclosed herein addresses those deficiencies of conventional cell culture medium comprising artificial and/or exogenous component by using human plasma to culture human organ cells, such as human hepatocytes.

### SUMMARY OF THE INVENTION

Herein are provided a method of culturing human organ cell, a cell culture system comprising human plasma culture medium and human organ cells and methods for using that system for evaluating test compound properties, liver disease progression or cell biology processes. In embodiments are provided methods of culturing human organ cells, wherein the method comprises the steps of culturing the human organ cells in a non-human plasma culture medium; replacing the non-human plasma culture medium with human plasma medium; and culturing the human organ cells in human plasma medium wherein the human plasma medium is refreshed with new human plasma medium at least once every, one (1) to fifteen (15) days or longer. In embodiments, the human plasma medium is replaced about every 2 to 3 days to replenish the nutrients and remove waste products.

The human organ cells may be cultured in the human plasma medium for an extended period of time and provides an improvement as compared to the length of time those human organ cells can be cultured in non-human plasma cell culture medium. See Example 1. In embodiments, the human organ cells are cultured in human plasma medium for at least 15 days, at least 20 days, at least 25 days, at least 30 days, at least 45 day, at least 60 days or at least 90 days. That is an improvement to culturing human organ cells (e.g., human hepatocytes) in non-human plasma medium wherein the cells start to disintegrate by day 14 and are nearly all dead at day 29. See FIG. 1. The cell culture methods disclosed herein provide a cell culture system that can be used in evaluation assays for periods of time beyond two weeks, such as 15 days or more. In embodiments, the human organ cells are cultured in human plasma medium up to 15 days, up to 20 days, up to 25 days, up to 30 days, up to 45 day, up to 60 days or up to 90 days, wherein the human organ cells remain viable during the culture period. Human hepatocytes are particularly useful for long term in vitro metabolism studies that can be translated to understanding human in vivo liver metabolism.

In embodiments, the human organ cells are freshly isolated, previously cryopreserved or obtained from a continuously cultured cell line. If previously cryopreserved, the methods comprise first thawing the cells under normal culture conditions, and then culturing with non-human plasma cell culture medium for a time period. That time period is typically at least 20 minutes, but may be a few hours to a day or more.

In embodiments, the human plasma medium consists of 100% plasma. In certain embodiments, the human plasma medium comprises 70% to 100% human plasma, or about 80% to 100% human plasma, or about 90% to about 100% human plasma, or about 95% to about 100% human plasma. In embodiments, the human plasma medium comprises about 70%, about 75%, about 80%, about 85%, about 90%, or about 95% human plasma.

In embodiments, the human organ cells are selected from hepatocytes, renal cells, pulmonary epithelial cells, enterocytes, cardiomyocytes, vascular endothelial cells, skeletal muscle cells, smooth muscle cells, neurons, lymphocytes, red blood cells, keratinocytes, adipocytes, adrenal cells, thyroid cells, thymic cells, connective tissue cells, testicular cells, ovarian cells, or a combination thereof. In certain embodiments, the human organ cells are human hepatocytes.

In embodiments, are provided methods for culturing human hepatocytes, wherein the methods comprise the steps of culturing the human hepatocytes in a non-human plasma culture medium; replacing the non-human plasma culture medium with human plasma medium; and, culturing the human organ cells in human plasma medium wherein the human plasma medium is refreshed with new human plasma medium at least once every, one (1) to fifteen (15) days or longer.

In embodiments, the human hepatocytes are cultured in human plasma medium for at least 15 days, at least 20 days, at least 25 days, at least 30 days, at least 45 day, at least 60 days or at least 90 days. In embodiments, the human hepatocytes are cultured in human plasma medium up to 15 days, up to 20 days, up to 25 days, up to 30 days, up to 45 day, up to 60 days or up to 90 days, wherein the human hepatocytes remain viable during the culture period.

In embodiments, the human hepatocytes are cultured in human plasma medium that consists of 100% plasma. In certain embodiments, the human hepatocytes are cultured in human plasma medium that comprises 70% to 100% human plasma, or about 80% to 100% human plasma, or about 90% to about 100% human plasma, or about 95% to about 100% human plasma. In embodiments, the human hepatocytes are cultured in human plasma medium that comprises about 70%, about 75%, about 80%, about 85%, about 90%, or about 95% human plasma.

In embodiments, the human hepatocytes are freshly isolated (from a donor), previously cryopreserved or obtained from a continuously cultured cell line. If previously cryopreserved, the methods comprise first thawing the human hepatocytes under normal culture conditions, and then culturing with non-human plasma cell culture medium for a time period. That time period is typically at least 20 minutes, but may be a few hours to a day or more.

For freshly isolated human hepatocytes, a fragment of a liver from a donor can be digested with collagenase, leading to dissociation of the liver parenchyma to individual hepatocytes which can be purified and cultured for various applications in human plasma medium. Freshly isolated human hepatocytes are not as convenient but are also routinely used.

In embodiments, are provided methods for culturing human hepatocytes, wherein the methods comprise the steps of isolating human hepatocytes from a human liver; culturing the human hepatocytes in a non-human plasma culture medium; replacing the non-human plasma culture medium with human plasma medium; and, culturing the human organ cells in human plasma medium wherein the human plasma medium is refreshed with new human plasma medium at least once every, one (1) to fifteen (15) days or longer.

In embodiments, the human hepatocytes isolated from a human liver are cultured in human plasma medium for at least 15 days, at least 20 days, at least 25 days, at least 30 days, at least 45 day, at least 60 days or at least 90 days. In embodiments, the human hepatocytes isolated from a human liver are cultured in human plasma medium up to 15 days, up to 20 days, up to 25 days, up to 30 days, up to 45 day, up to 60 days or up to 90 days, wherein the human hepatocytes remain viable during the culture period.

In embodiments, the human hepatocytes are cultured in human plasma medium that consists of 100% plasma. In certain embodiments, the human hepatocytes are cultured in human plasma medium that comprises 70% to 100% human plasma, or about 80% to 100% human plasma, or about 90% to about 100% human plasma, or about 95% to about 100% human plasma. In embodiments, the human hepatocytes are cultured in human plasma medium that comprises about 70%, about 75%, about 80%, about 85%, about 90%, or about 95% human plasma.

In embodiments, is provided a cell culture system for evaluating test compound properties, liver disease progression or cell biology processes, wherein the cell culture system comprises a cell culture vessel comprising one or more assay wells, wherein the assay wells comprise human plasma culture medium and human organ cells. In embodiments, the cell culture system further comprises a virus, such as a hepatitis virus. In embodiments, the cell culture system further comprises a test compound. The test compound may be selected from a drug, a drug candidate, an industrial chemical, an environmental pollutant, a pesticide, an insecticide, a biological chemical, and a chemical.

In embodiments, the test compound properties comprise drug metabolism, drug-drug interactions, and pharmacology of the test compound. In embodiments, the liver disease comprises hepatitis A infection, hepatitis B infection or hepatitis C infection. In embodiments, the cell biology processes comprise gene expression, protein synthesis or response to hormones.

In embodiments, the cell culture system comprises human organ cells that are freshly isolated (from a donor), previously cryopreserved or obtained from a continuously cultured cell line. In embodiments, the cell culture system comprises human plasma medium that consists of 100% plasma. In certain embodiments, the cell culture system comprises human plasma medium that comprises 70% to 100% human plasma, or about 80% to 100% human plasma, or about 90% to about 100% human plasma, or about 95% to about 100% human plasma. In embodiments, the cell culture system comprises human plasma medium that comprises about 70%, about 75%, about 80%, about 85%, about 90%, or about 95% human plasma. In embodiments, the human plasma is prepared from blood obtained from human donors. In certain embodiments, the human plasma is pooled from multiple donors.

In embodiments, the cell culture system comprises human organ cells selected from hepatocytes, renal cells, pulmonary epithelial cells, enterocytes, cardiomyocytes, vascular endothelial cells, skeletal muscle cells, smooth muscle cells, neurons, lymphocytes, red blood cells, keratinocytes, adipocytes, adrenal cells, thyroid cells, thymic cells, connective tissue cells, testicular cells, ovarian cells, or a combination thereof. In certain embodiments, the cell culture system comprises human hepatocytes.

In embodiments, the cell culture system comprises a cell culture vessel that is a single well plate. In alternative embodiments, the cell culture system comprises a cell culture vessel that is a multi-well plate.

In embodiments, is provided a cell culture system for evaluating test compound properties, liver disease progression or cell biology processes, wherein the cell culture system comprises a cell culture vessel comprising one or more assay wells, wherein the assay wells comprise human plasma culture medium and human hepatocytes. In embodiments, the cell culture system comprises human hepatocytes and further comprises a virus, such as a hepatitis virus. In embodiments, the cell culture system comprises human hepatocytes and further comprises a test compound. The test compound may be selected from a drug, a drug candidate, an industrial chemical, an environmental pollutant, a pesticide, an insecticide, a biological chemical, and a chemical.

In embodiments, the test compound properties comprise drug metabolism, drug-drug interactions, and pharmacology of the test compound. In embodiments, the liver disease comprises hepatitis A infection, hepatitis B infection or hepatitis C infection. In embodiments, the cell biology processes comprise gene expression, protein synthesis or response to hormones.

In embodiments, the cell culture system comprises human hepatocytes that are freshly isolated from a liver, previously cryopreserved or obtained from a continuously cultured cell line. In embodiments, the cell culture system comprises human hepatocytes and human plasma medium that consists of 100% plasma. In certain embodiments, the cell culture system comprises human hepatocytes and human plasma medium that comprises 70% to 100% human plasma, or about 80% to 100% human plasma, or about 90% to about 100% human plasma, or about 95% to about 100% human plasma. In embodiments, the cell culture system comprises human hepatocytes and human plasma medium that comprises about 70%, about 75%, about 80%, about 85%, about 90%, or about 95% human plasma.

In embodiments, are provided methods for evaluating test compound properties, liver disease progression and/or cell biology processes, wherein the method comprises the steps of introducing a test compound into an assay well of a present cell culture system, incubating the test compound for 0.5 h to 10 days at 33 to 40° C.; and, performing an end point assay of the human cells or cell culture medium to determine properties of the test compound, disease progression or cell biology processes. In embodiments, the system is incubated at 37° C. at 5% CO₂.

In embodiments, the test compound is a drug, a drug candidate, an industrial chemical, an environmental pollutant, a pesticide, an insecticide, a biological chemical, or a chemical. In embodiments, the test compound properties comprise drug metabolism, drug-drug interactions, and pharmacology of the test compound.

In alternative embodiments, methods are provided herein where a test compound is not added to the cell culture system. In embodiments, the methods comprise adding a virus but into a test compound to the cell culture system. In certain embodiments, the methods comprise adding a virus to the cell culture system followed by addition of a test compound to the cell culture system. In embodiments, the end point assay comprises measuring cell viability, cell function, gene expression, protein expression, metabolite formation or metabolite profiles. In certain embodiments, the end point assay comprises measuring disappearance of a test compound and the appearance of metabolites, measuring effect of a first test compound on metabolism of co-administered second test compound, or measuring pharmacological effects on the human organ cells.

In methods for evaluating liver disease progression, an end point assay of the human hepatocytes or cell culture medium is performed for evaluation of cell biology, chemistry and/or molecular biology of the human hepatocytes relevant to the disease progression. In embodiments, liver disease comprises hepatitis A infection, hepatitis B infection or hepatitis C infection. In embodiments, the methods for evaluating liver disease comprise use of a cell culture system comprising human hepatocytes, and addition of a virus. In embodiments, the virus is hepatitis A virus, hepatitis B virus or hepatitis C virus. In certain embodiments the methods for evaluating liver disease progression further comprises adding a test compound after addition of the virus to the cell culture system.

In methods for evaluating cell biology process, for example in response to a test compound or a disease progression, an end point assay of the human hepatocytes or cell culture medium is performed for evaluation of cell biology, chemistry and/or molecular biology of the human hepatocytes relevant to the cell biology processes being investigated.

### BRIEF DESCRIPTION OF THE DRAWINGS

FIG. 1 provides cell morphology of human hepatocytes from a donor cultured in William E medium and human plasma medium at day 7, 14 and 24. The human hepatocytes cultured in William's E medium started to disintegrate at day 14 and continued to disintegrate to Day 24. The human hepatocytes cultured in human plasma medium remained intact with excellent epithelial cell morphology up to day 24.

FIG. 2 provides morphology of human hepatocytes cultured in human plasma medium and William's E medium for 29 days. The human hepatocytes cultured in William's E medium are nearly all dead while the human hepatocytes show excellent cell morphology.

FIG. 3 provides cell morphology of human hepatocytes cultured in human plasma medium and protein fee hepatocyte media (HIM) at day 7, 14, 24 and 29.

FIG. 4 provides a table of the fold increase in CYP enzyme induction (as measured by mRNA) of CYP1A2 (by omeprazole), CYP2B6 (by phenobarbital) and CYP2A4 (by rifampin) from human hepatocytes cultured in human plasma medium or HIM.

FIG. 5 provides evaluation of acute and subchronic hepatotoxicity from human hepatocyte cultured in human plasma medium in vitro with Aflatoxin B1, which requires metabolic activation for hepatoxicity. FIG. 5 demonstrates Aflatoxin B1 was cytotoxic to human hepatocytes cultured in human plasma medium.

FIG. 6 provides a dose dependent rifampin induction of CYP3A4 gene expression, as measured by mRNA, in human hepatocytes from a single donor cultured in human plasma medium or HIM.

FIG. 7 provides a dose dependent rifampin induction of CYP3A4 gene expression, as measured by mRNA, in human hepatocytes from a single donor cultured in human plasma medium or HIM.

### DETAILED DESCRIPTION OF THE INVENTION

Provided herein are methods, compositions, and kits for culturing human organ cells in human plasma medium, a cell culture system comprising the human organ cells and human plasma in cell culture vessel, and use of the cultured human organ cells for evaluation of test compound properties, organ disease progression and/or cell biology processes. In embodiments, the methods include methods for culturing human organ cells in human plasma medium comprising 70% to 100% human plasma. In certain embodiments, the methods comprise culturing human organ cells in a non-human plasma culture medium, replacing the non-human plasma culture medium with human plasma medium and culturing the human organ cells in human plasma medium wherein the human plasma medium is refreshed with new human plasma medium at least once every one (1) to fifteen (15) days or long. In embodiments, the human plasma medium is changed to fresh human plasma medium, e.g. every 2-3 days, to allow replenishment of nutrient and removal of waste products. In certain embodiments, the human organ cells are human hepatocytes. In other certain embodiments, the human organ cells are hepatocytes cultured in 100% human plasma medium.

The methods herein are carried out by culturing the human organ cells in human plasma medium for an extended period of time, for example from 1 to about 30 days, or longer (e.g. up to about 90 days). The length of culture will depend on their use in methods for evaluating test compound proprieties, organ disease progression and/or cell biology processes wherein one of skill in the art understands the duration of cell culture needed for a particular assay. In embodiments, the human organ cells in human plasma medium are cultured for at least 15 days. In other embodiments, the human organ cells in human plasma medium are cultured for up to about 15 days. In embodiments, the human organ cells are cultured in human plasma medium for at least 15 days, at least 20 days, at least 25 days, at least 30 days, at least 45 day, at least 60 days or at least 90 days. In embodiments, the human organ cells are cultured in human plasma medium up to 15 days, up to 20 days, up to 25 days, up to 30 days, up to 45 day, up to 60 days or up to 90 days, wherein the human organ cells remain viable during the culture period.

In embodiments, the human plasma medium consists of 100% plasma. In certain embodiments, the human plasma medium comprises about 70% to 100% human plasma, or about 80% to 100% human plasma, or about 90% to about 100% human plasma, or about 95% to about 100% human plasma. In embodiments, the human plasma medium comprises about 70%, about 75%, about 80%, about 85%, about 90%, or about 95% human plasma.

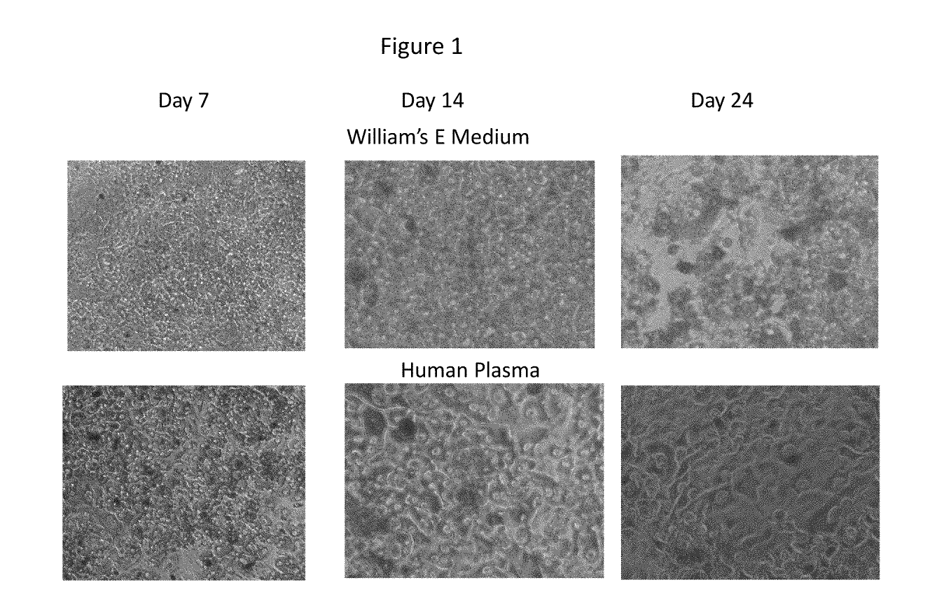

The methods herein, culturing human organ cells in human plasma medium, provide human organ cells that survive, with excellent cell morphology, days and weeks longer than if those same cells were cultured in non-human plasma growth cell culture medium. See Example 1 and FIGS. 1-3. When human hepatocytes are cultured in a non-human cell culture medium, such as William's E medium, the cells start to disintegrate by day 14 and are nearly all dead within a month at day 29. In contrast, the human hepatocytes cultured in human plasma medium are viable with excellent cell morphology after a month of cell culture wherein the human plasma medium is changed every 2 to 3 days. That cell culture methods provide an improved cell culture system for evaluating test compound properties, organ disease progression and/or cell biology processes.

In embodiments, the compositions comprise a cell culture system for evaluating test compound properties, organ disease progression (such as liver disease) and/or cell biology processes comprising a cell culture vessel comprising one or more assay wells, wherein the assay wells comprise human plasma culture medium and human organ cells. In certain embodiments, the cell culture system is used for high-throughput screening to test the metabolic effects or response to a range of test compounds. In that instance, the cell culture system comprises a cell culture vessel that is a multi-well plate, such as a 6-well; 12-well; 24-well; 48-well, 96-well; 384-well, 1536-well plate or any combination thereof. In alternative embodiments, the exogenous metabolic system comprises a cell culture vessel with a single assay well.

In certain embodiments, use of the cell culture system herein is carried out by the addition of a test compound or test article to the cell culture system. The test compound is introduced into the assay well, incubated and then an end point assay of the human organ cell or cell culture medium is performed to determine the properties of the test compound including cell biology processes impacted by the test compound. In embodiments, the test compound properties comprise drug metabolism, drug-drug interactions, and pharmacology of the test compound. In alternative embodiments, no test compound is introduced into the cell culture system. In that instance, the human organ cells are cultured in human plasma medium and an end point assay of the human organ cells or cell culture medium is performed for evaluation of cell biology, chemistry and molecular biology relevant to the cell biology processes or organ disease progression being evaluated. In embodiments, the organ disease is liver disease such as hepatitis A, hepatitis B infection or hepatitis C infection. In embodiments, the cell biology processes comprise gene expression, protein synthesis or response to hormones.

In certain embodiments, use of the cell culture system herein is carried out by the addition of a virus to the cell culture system evaluation of disease progression. In embodiments, a hepatitis virus is added to a cell culture system comprising human hepatocytes and human plasma medium. In embodiments, a test compound is subsequently added to the cell culture system comprising human hepatocytes, human plasma medium and hepatitis virus. In embodiments, an end point assay of the human hepatocytes or cell culture medium is performed for evaluation of cell biology, chemistry and molecular biology relevant to the cell biology processes or liver disease progression being evaluated.

The test compounds used in the present invention include, but are not limited to, drugs, drug candidates, biologicals, food components, herb or plant components, proteins, peptides, oligonucleotides, DNA and RNA. In embodiments, the test compound is a drug, a drug candidate, an industrial chemical, an environmental pollutant, a pesticide, an insecticide, a biological chemical, a vaccine preparation, a cytotoxic chemical, a mutagen, a carcinogen, a hormone, an inhibitory compound, a chemotherapeutic agent or a chemical. The test compound can be either naturally-occurring or synthetic, and can be organic or inorganic. A person skilled in the art will recognize that the test compound can be added to the human plasma medium present in the cell culture system in an appropriate solvent or buffer.

In embodiments, the test compound is a drug, a drug candidate, an industrial chemical, an environmental pollutant, a pesticide, an insecticide, a biological chemical, or a chemical

In embodiments, the human organ cells are selected from hepatocytes, renal cells, pulmonary epithelial cells, enterocytes, cardiomyocytes, vascular endothelial cells, skeletal muscle cells, smooth muscle cells, neurons, lymphocytes, red blood cells, keratinocytes, adipocytes, adrenal cells, thyroid cells, thymic cells, connective tissue cells, testicular cells, ovarian cells, or a combination thereof. In certain embodiments, the human organ cells are human hepatocytes that are cultured in human plasma medium. It is understood that reference to “human organ cells” means any of the above individual organ cells such as hepatocytes or enterocytes, as if they were specifically disclosed in the relevant text.

In embodiments, the human organ cells cultured in human plasma medium are freshly isolated, previously cryopreserved, or obtained from a continuously cultured cell line. Therefore, in embodiments, prior to culturing human organ cells in a non-human plasma culture medium, the organ cells may first be thawed and cultured in a culture medium, such as a non-human plasma culture medium. In certain embodiments, the organ cells can be cultured in the culture medium for a time period sufficient for the organ cells to attach to a surface of a cell culture vessel. In embodiments, the time period is at least about 20 minutes, but may be up to four (4) hours or longer. In certain embodiments, the human organ cells are human hepatocytes that were previously cryopreserved from donors, which may be cryopreserved as an individual lot from one donor or as a pool from multiple donors.

In embodiments, the human organ cells are primary cells or cell lines, cryopreserved or freshly prepared. In certain embodiments, the human organ cells are from organs responsible for drug metabolism in vivo, such as liver (hepatocytes), intestine (enterocytes) and kidney (renal cells). In embodiments, the human organ cells are hepatocytes for hepatic metabolism, enterocytes for intestinal metabolism or renal proximal tube cells for renal metabolism. In certain embodiments, the different organ cells are used individually to define the role of each organ on the metabolism of the test compound. In other words, enterocytes can be used to model enteric metabolism for orally ingested test compounds, pulmonary epithelia cells to model metabolism of inhaled test compounds and renal proximal tubule cells can be used to model renal metabolism for renal excreted test compounds. In alternative embodiments, the different organ cells are used in combination to understand the overall metabolism of a test compound. In certain embodiments, the test compound determines the type of human organ cells used in the present cell culture system. In embodiments, for a test compound wherein oral is the primary route of administration enterocytes may be selected as the human organ cells. Alternatively, if the test compound is renal-excreted, then renal proximal tubule cells may be selected as the human organ cells. Accordingly, the cell culture system comprises a cell culture vessel, human plasma medium and human hepatocytes, enterocytes or renal proximal tubule cells.

In certain embodiments, the human organ cells are human hepatocytes. See, Li, A. P. (2007) Human hepatocytes: isolation, cryopreservation and applications in drug development. Chemico-biological interactions, 168(1), 16-29. Freshly prepared human organ cells or cryopreserved cells (e.g. hepatocytes) obtained by standard methods from a human liver can be used as the human organ cells. See, Hewitt, Nicola J., et al. “Primary hepatocytes: current understanding of the regulation of metabolic enzymes and transporter proteins, and pharmaceutical practice for the use of hepatocytes in metabolism, enzyme induction, transporter, clearance, and hepatotoxicity studies.” Drug metabolism reviews 39.1 (2007): 159-234. The hepatocytes can be used in the form of suspensions or plated on a suitable culture plate containing appropriate growth medium (e.g. non-human plasma culture medium).

One of skill in the art understands how to select an appropriate non-human plasma cell culture growth medium. For hepatocytes, cell culture growth medium includes, but is not limited to, William's E Medium and Dulbecco's Modified Eagle's Minimal Medium.

In embodiments, the human organ cells are hepatocytes (the parenchymal cells of the liver). Hepatic metabolism is known to be the major determinant of metabolism-dependent xenobiotic toxicity. P450 and non-P450 phase 1 oxidation enzyme pathways are responsible mostly for the bio activation of relatively inert parent compounds to reactive (toxic/carcinogenic/mutagenic) metabolites. Phase 2 conjugating pathways are responsible mostly for the biotransformation of toxic parent compounds or metabolites to less toxic compounds. Both phase 1 and phase 2 pathways are present in the hepatocytes—the key hepatic cell type responsible for hepatic metabolism. In embodiments, enterocytes can be used to model enteric metabolism for orally ingested toxicants. In other embodiments, renal proximal tubule cells can be used to model renal metabolism. In certain other embodiments, enterocytes can be used to model intestinal metabolism.

In certain embodiments, the human organ cells are recombinant cells, wherein for example, they have been engineered to comprise drug metabolizing enzymes. In certain embodiments, the human organ cells are engineered to contain cytochrome P450 isoforms. See, Doehmer, J., Battula, N., Wölfel, C., Kudla, C., Keita, Y. and Staib, A. H., 1992. Biotransformation of caffeine and theophylline in mammalian cell lines genetically engineered for expression of single cytochrome P450 isoforms. Biochemical pharmacology, 43(2), pp. 225-235; Donato, M. T., Jiménez, N., Castell, J. V., & Gómez-Lechón, M. J. (2004). Fluorescence-based assays for screening nine cytochrome P450 (P450) activities in intact cells expressing individual human P450 enzymes. Drug Metabolism and Disposition, 32(7), 699-706.

In embodiments, a stable cell line such as HepG2 is cultured with human plasma medium, used in the present cell culture system and methods for determining metabolism of a test compound. HepG2 is a perpetual cell line derived from the liver of a 15-year-old Caucasian male with a well-differentiated hepatocellular carcinoma. Because of the high degree of morphological and functional differentiation in vitro, HepG2 cells can be a suitable model to study the intracellular trafficking and dynamics of bile canalicular and sinusoidal membrane proteins and lipids in human hepatocytes in vitro. See, Ihrke et al., WIF-B cells: an in vitro model for studies of hepatocyte polarity. Journal of Cell Biology 123 (6), 1761-1775, 1993. In certain embodiments, a stable cell line such as HepaRG is cultured with human plasma medium, used in the present cell culture system and methods for determining metabolism of a test compound. See, Guillouzo, A., Corlu, A., Aninat, C., Glaise, D., Morel, F. and Guguen-Guillouzo, C., 2007. The human hepatoma HepaRG cells: a highly-differentiated model for studies of liver metabolism and toxicity of xenobiotics. Chemico-biological interactions, 168(1), pp. 66-73.

In embodiments, present human organ cells may be genetically altered or modified so as to contain a non-native “recombinant” (also called “exogenous”) nucleic acid sequence, or modified by antisense technology to provide a gain or loss of genetic function. Methods for generating genetically modified cells are known in the art, see for example “Current Protocols in Molecular Biology,” Ausubel et al., eds, John Wiley & Sons, New York, N.Y., 2000.

Accordingly, in embodiments, the methods for culturing human organ cells with human plasma medium is suitable for use with any organ cell type, including primary cells, stem cells, progenitor cells, normal, genetically-modified, genetically altered, immortalized, and transformed cell lines. The present invention is suitable for use with single cell types or cell lines, or with combinations of different cell types. The cell may be of any tissue type (e.g., heart, stomach, kidney, intestine, lung, liver, fat, bone, cartilage, skeletal muscle, smooth muscle, cardiac muscle, bone marrow, muscle, brain, pancreas), and cell type (e.g., epithelial, endothelial, mesenchymal, adipocyte, hematopoietic).

In embodiments, the compositions comprise a cell culture system for evaluating test compound properties, liver disease progression or cell biology processes comprising a cell culture vessel comprising one or more assay wells, wherein the assay wells comprise human plasma culture medium and human organ cells. In embodiments, the cell culture system is used wherein a test compound is introduced into the assay well, the test compound is incubated 0.5 h to 10 days at 33-40° C. and then an end point analysis of the human cells or cell culture medium to determine properties of the test compound is performed. The human organ cells cultured in human plasma medium can be used for the evaluation of drug pharmacological activities, drug metabolism, drug-drug interactions, and drug toxicity, with results that can be translated directly to human in vivo.

In embodiments, cells are cultured from a variety of genetically diverse individuals who may respond differently to biologic and pharmacologic agents. Genetic diversity can have indirect and direct effects on metabolism of a test compound. In embodiments, the human organ cells are a pool of cells from multiple individuals or donors. In certain embodiments, the human organ cells are reflective of the heterogeneity of a population of individuals.

In embodiments, the human organ cells are involved in the detoxification and metabolism of pharmaceutically active compounds, e.g., liver cells, including hepatocytes; kidney cells including tubule cells; fat cells including adipocytes that can retain organic compounds for long periods of time. In embodiments, these cells can be combined in a cell culture system with cells such as lung cells, which are involved in respiration and oxygenation processes.

In embodiments, the human organ cells, either previously cryopreserved, freshly isolated from a donor organ or from a continuously cultured cell line, are cultured in a non-human plasma cell culture medium, which is replaced with human plasma medium, for at least 20 minutes and up to a few hours or a day or more. The human plasma medium comprises from about 70% to 100% human plasma. In certain embodiments, the human plasma medium consists of 100% human plasma. In certain embodiments, the human plasma medium may comprise of about 70%, about 75%, about 80%, about 85%, about 90% or about 95% human plasma, with the balance being routinely used cell culture medium. In certain embodiments, human organ cells are cultured in human plasma medium comprising about 75% to 100% human plasma, about 80% to 100% human plasma, about 85% to 100% human plasma, about 90% to 100% human plasma and about 95% to 100% human plasma. The human plasma may be pooled from multiple donors, or from an individual donor. In embodiments, the human plasma is pooled from at least 5 donors. In embodiments, the human plasma is obtained from human blood. See Example 1 wherein human hepatocytes were cultured from 29 days in 100% human plasma medium, with a culture medium change every 2-3 days, and show excellent cell morphology at day 29. In comparison, human hepatocytes cultured in a non-human plasma medium, such as William's E medium, showed some disintegrate at day 14 and were nearly all dead at day 29.

In embodiments, the human organ cells in human plasma medium may be cultured for at least 15 days, at least 20 days, at least 29 days, at least 30 days, at least 45 days, at least 60 days or at least 90 days. In certain embodiments, the human organ cells in human plasma medium may be cultured for at least one day, at least 2 days, at least 5 days, at least 10 days, or at least 15 days. In certain embodiments, the human organ cells in human plasma medium may be cultured up to 5 days, up to 10 days, up to 15 days, up to about 30 days, up to 45 days, up to 60 days or up to 90 days.

In certain embodiments, human hepatocytes are cultured in human plasma medium wherein the steps comprise culturing the human hepatocytes in a non-human plasma culture medium; replacing the non-human plasma culture medium with human plasma medium; and, culturing the human organ cells in human plasma medium wherein the human plasma medium is refreshed with new human plasma medium at least once every, one (1) to fifteen (15) days or longer. In embodiments, the human hepatocytes are cryopreserved, thawed and then cultured in the non-human plasma medium. In other embodiments, human hepatocytes are cultured in human plasma medium wherein the steps comprise isolating human hepatocytes from a human liver; culturing the human hepatocytes in a non-human plasma culture medium; replacing the non-human plasma culture medium with human plasma medium; and, culturing the human organ cells in human plasma medium wherein the human plasma medium is refreshed with new human plasma medium at least once every, one (1) to fifteen (15) days or longer. In embodiments, the human hepatocytes are cultured in human plasma medium for at least one week, two weeks, three weeks or at least about a month (e.g., four weeks or about 29-31 days). In embodiments, the cell culture conditions replicate physiological conditions as much as possible. The term “physiological conditions” as used herein is defined to mean that the cell culturing conditions are very specifically monitored to mimic as closely as possible the natural tissue conditions for a particular type of cell in vivo.

In embodiments are provided use of the cell culture system comprising human organ cells and human plasma medium in a cell culture vessel, for evaluation of test compound properties, organ disease progression and/or cell biology processes.

In embodiments, the test compound is considered an input variable, and is used interchangeably herein with a test compound, of the cell culture system. The test compounds are screened for biological activity by adding to a pharmacokinetic-based culture system (e.g. present exogenous metabolic system), and then assessing the target cells (or culture medium) for changes in output variables of interest, e.g., consumption of O₂, production of CO₂, cell viability, expression of proteins of interest (protein expression), cell function, expression of genes of interest (gene expression), metabolite formation or metabolite profiles. The test compound is typically added in solution, or readily soluble form, to the human plasma medium of cells in culture. The test compound can be added using a flow through system, or alternatively, adding a bolus to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall composition of the culture medium should not change significantly with the addition of the bolus, or between the two solutions in a flow through method.

In embodiments, the test compound includes pharmacologically active drugs or drug candidates and genetically active molecules. Test compounds of interest include chemotherapeutic agents, anti-inflammatory agents, hormones or hormone antagonists, ion channel modifiers, and neuroactive agents. Exemplary of pharmaceutical agents suitable for this invention are those described in “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology. Also included are toxins, and biological and chemical warfare agents, for example see Somani, S. M. (Ed.), “Chemical Warfare Agents,” Academic Press, New York, 1992).

In embodiments, the test compound includes all of the classes of molecules disclosed herein, and may further or separately comprise samples of unknown content. While many samples will comprise compounds in solution, solid samples that can be dissolved in a suitable solvent may also be assayed. Samples containing test compounds of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include test compounds being assessed for potential therapeutic value, e.g., drug candidates from plant or fungal cells.

Test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

In embodiments, following incubation of the cell culture system with the test compound an end point analysis is performed to determine properties of the test compound including disease progression and cell biology processes. In embodiments, the end point analysis identifies the output variable (e.g. property of the test compound) of the cell culture system. In embodiments, output variables are quantifiable elements of the cells, particularly elements that can be accurately measured in a cell culture system. An output variable can be any cell component or cell product including, e.g., viability, respiration, metabolism, cell surface determinant, receptor, protein or conformational or posttranslational modification thereof, lipid, carbohydrate, organic or inorganic molecule, mRNA, DNA, or a portion derived from such a cell component. In embodiments, the output variable is directly or indirectly a result of the test compound or its metabolite. While most output variables will provide a quantitative readout, in some instances a semi-quantitative or qualitative result will be obtained. Readouts may include a single determined value, or may include mean, median value or the variance. Characteristically a range of readout values will be obtained for each output. Variability is expected and a range of values for a set of test outputs can be established using standard statistical methods.

Various methods can be utilized for quantifying the presence of selected metabolism markers. Liquid chromatography (LC), mass spectrometry (MS), and their combination (LC/MS-MS) are routinely used for the quantification of metabolites. For non-LC/MS measurement of the amount of a molecule that is present, a convenient method is to label the molecule with a detectable moiety, which may be fluorescent, luminescent, radioactive, or enzymatically active. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g., by expressing them as green fluorescent protein chimeras inside cells (for a review, See Jones et al. (1999) Trends Biotechnol. 17(12):477-81).

Output variables may be measured by immunoassay techniques such as, immunohistochemistry, radioimmunoassay (RIA) or enzyme linked immunosorbance assay (ELISA) and related non-enzymatic techniques. These techniques utilize specific antibodies as reporter molecules that are particularly useful due to their high degree of specificity for attaching to a single molecular target. Cell based ELISA or related non-enzymatic or fluorescence-based methods enable measurement of cell surface parameters. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these. For toxicity assays, outputs can include measurement of cell viability such as enzyme release, cellular ATP contents, reactive oxygen species formation, decrease of reduced glutathione, protein synthesis, protein contents, DNA contents, dye exclusion, dye inclusion, and cell detachment. For pharmacological assays, specific disease target related assays can be used.

In embodiments, the results of screening assays may be compared to results obtained from a reference compound, concentration curves, controls (with and without metabolically competent cells), etc. The comparison of results is accomplished by the use of suitable deduction protocols, AI systems, statistical comparisons, algorithms, etc.

A database of reference output data can be compiled. These databases may include results from known agents or combinations of agents, as well as references from the analysis of cells treated under environmental conditions in which single or multiple environmental conditions or parameters are removed or specifically altered. A data matrix may be generated, where each point of the data matrix corresponds to a readout from an output variable, where data for each output may come from replicate determinations, e.g., multiple individual cells of the same type.

The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### EXAMPLES

The following examples are put forth so as to provide those of ordinary skill in the art with a complete disclosure and description of how to use the embodiments provided herein, and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Culturing of Human Hepatocytes in Whole (100%) Human Plasma

In the human body, all cells are nourished by plasma. Human plasma should therefore be the most appropriate medium for the culturing of human cells.

Previously cryopreserved 100% confluent plateable human hepatocytes (HH1053/57/62; three donors) were cultured in 100% human plasma (minimally modified human plasma pooled from five donors) and William's E medium (Thermo Fisher), which is a reduced serum-supplemented medium for long-term cell cultures of adult rat liver epithelial cells but can also be used for culturing human hepatocytes. The hepatocytes were cultured in a 96 well plates with the culture medium changed every Monday, Wednesday and Friday for a total of 29 days.

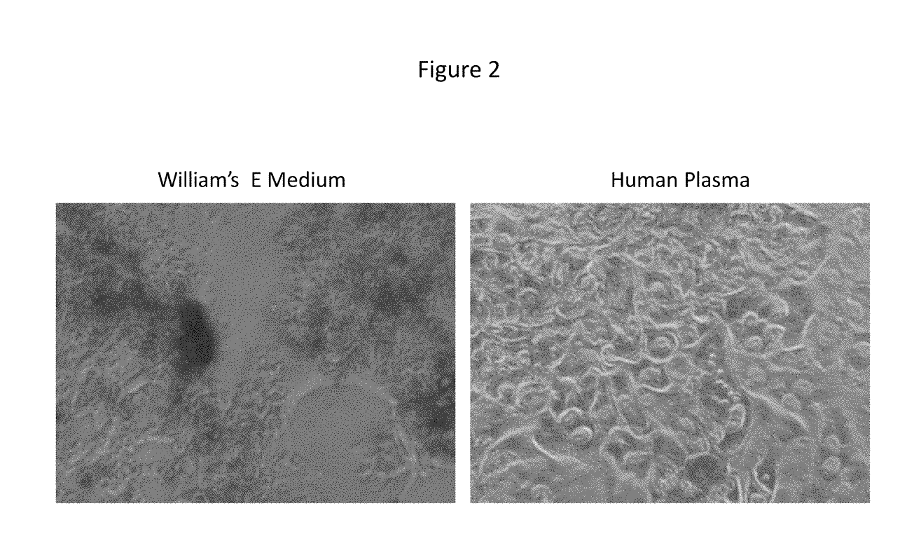

Cell morphology of the cultured hepatocytes was evaluated on day 7, 14, 24, and 29 wherein the human hepatocytes cultured in the William's E medium started to disintegrate at day 14 and continued through the end of the experiment at day 29. In contrast, the human hepatocytes cultured in the 100% human plasma medium remained intact with normal epithelial cell morphology through day 24. See FIG. 1. The human hepatocytes cultured in William's E medium were nearly all dead at day 29, while the human hepatocytes cultured in 100% human plasma remained viable with normal epithelial cell morphology. See FIG. 2.

Human hepatocytes can be cultured for an extended period (over 4 weeks) in human plasma. In contrast, human hepatocytes cultured in William's E medium, the most commonly used medium for hepatocyte culturing, the cells start to deteriorate at about two week or 14 days.

### Example 2: Cytochrome P450 Induction

Hepatocytes from human donors (previously cryopreserved) were plated in both 100% human plasma (minimally modified human plasma medium pooled from donors; HPZ-A) and protein free hepatocyte media (HIM) for a total duration of 29 days. The hepatocytes were plated in 96-well collagen-coated tissue culture plates using Universal Cryopreservation Plating Medium (UCPM). After 4 hours of attachment, the medium was changed to HPZ-A and HIM containing 0.25 mg/mL Matrigel. Medium was changed to HPZ-A without Matrigel on the next day and every 2-3 days afterward for a culture duration of 29 days.

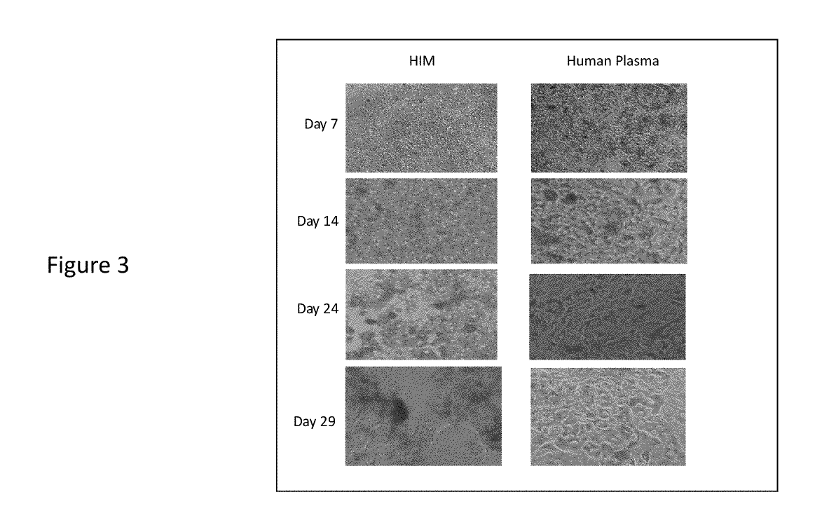

FIG. 3 shows the morphology of human hepatocytes cultured in human plasma medium as compared to HIM on day 7, 14, 24 and 29. The human hepatocytes cultured in human plasma medium remained viable with normal epithelial cell morphology at day 29. Hence, a 100% human plasma medium (HPZ-A) can be used successfully for culturing human hepatocytes for a prolonged period of time of at least 29 days or longer.

For CYP induction comparisons, 4 lots of human hepatocytes (HH1007; HH1026; HH1051; and HH1053) were used. The cytochrome P450 proteins are monooxygenases that catalyze many reactions involved in drug metabolism. The hepatocytes were plated in 96-well plates as disclosed above in both HIM and human plasma medium, with treatment prototypical inducers beginning on day 5 after plating. The treatment duration was for 3 days. Gene expression by RT-PCR was used to measure induction of CYP1A2 (omeprazole), CYP2B6 (by phenobarbital) and CYP3A4 (by rifampin).

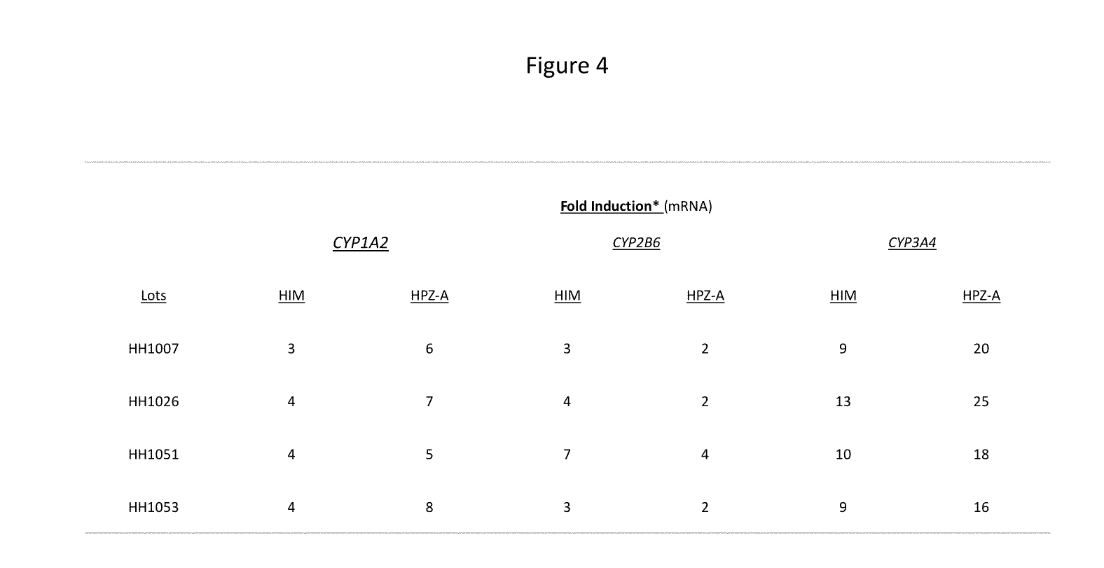

FIG. 4 shows induction of each of CYP1A2, CYP2B6 and CYP3A4 as measured by mRNA for each of the four lots of human hepatocytes cultured in both HIM and human plasma medium. The human hepatocytes cultured in human plasma medium are responsive to CYP1A2, CYP2B6 and CYP3A4 induction, demonstrating the retention of intact P450 induction pathways.

For long-term cytotoxicity, lot HH1062 was cultured in human plasma medium as disclosed above, and treated with seven concentrations of aflatoxin B1 (concentration ranged from 1 uM to 90 uM) on day 2 and day 10. Viability was quantified based on cellular ATP contents (ATPLite, Perkin-Elmer). Results were expressed as Relative Viability using the following equation:

Relative Viability(%)=ATP(Treatment)/ATP(Solvent Control)×100.

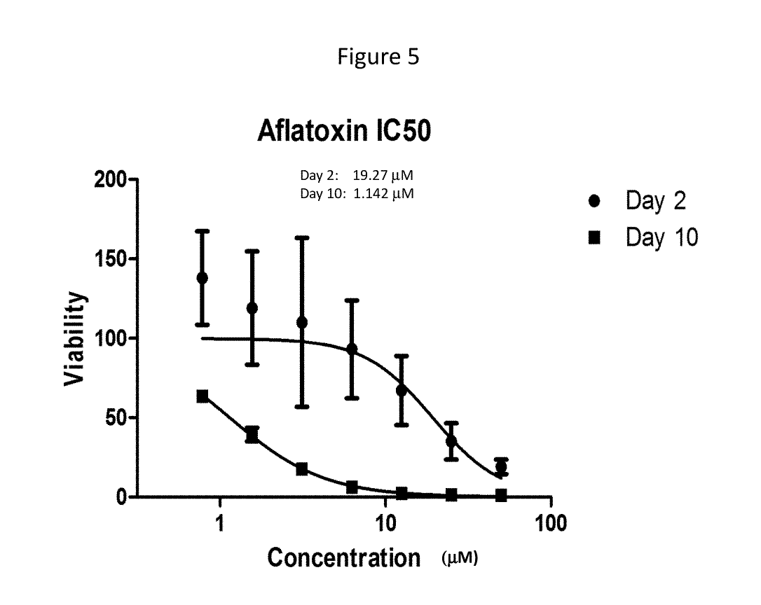

FIG. 5 shows in vitro acute and subchronic hepatotoxicity evaluation in human plasma. Aflatoxin B1, which requires metabolic activation for hepatotoxicity, was found to be cytotoxic to human hepatocytes cultured in human plasma medium. Aflatoxin UC50 value after a day 10 treatment duration was 19× lower than that observed after a 2-day treatment duration. The results indicate that human hepatocytes cultured in human plasma could activate a protoxicant and can be used for the evaluation of chronic hepatotoxicity.

### Example 3: Cytochrome P450 3A4 Induction

CYP3A4 is a member of the cytochrome P450 superfamily of enzymes.

Hepatocytes from two human donors (previously cryopreserved) were plated and cultured as disclosed in Example 2. For CYP3A4 induction comparisons, lots of human hepatocytes (HH1051 and HH1053) were used. For the hepatocytes cultured in HIM, rifampin (concentration ranged from 1 uM to 20 uM) was added at day 2 after plating for a treatment duration of 3 days and for the hepatocytes cultured in human plasma medium, rifampin (concentration ranged from 10 uM to 200 uM) was added at day 5 after plating for a treatment duration of 3 days. Gene expression by RT-PCR was used to measure induction of CYP3A4 by rifampin.

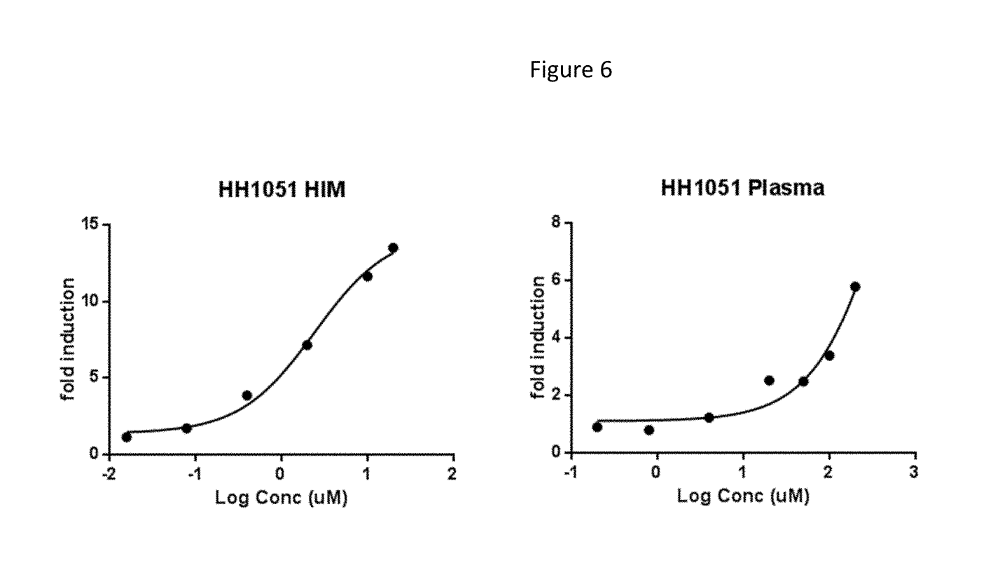

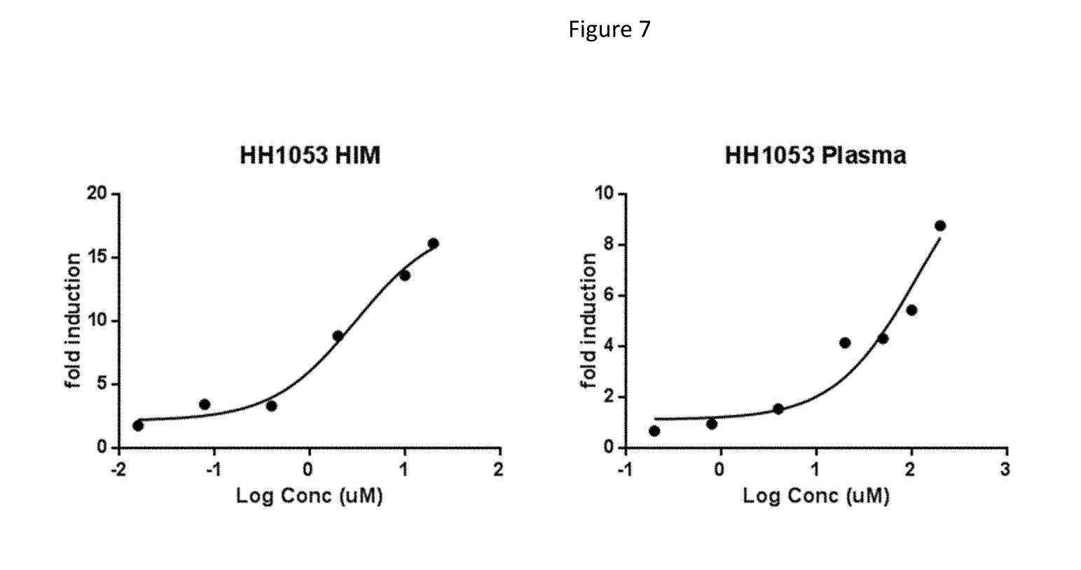

FIGS. 6 and 7 show a dose dependent induction of CYP3A4 gene expression by rifampin. At the concentrations evaluated, the maximal fold induction in the hepatocytes cultured in human plasma medium was found to be lower than that observed by the hepatocytes cultured in HIM.

### Example 4: Evaluation of Liver Disease Mechanism and Cure

A potential application of human hepatocytes cultured in human plasma is for the evaluation of hepatitis A, B, and/or C. Human plasma hepatocyte cultures can be infected with either purified viruses or virus-containing plasma (e.g. from hepatitis patients). Viral replication and resulting changes in the hepatocytes can aid the understanding of the mechanism of viral infection and hepatic damage. The virally infected hepatocytes can also be used to screen for anti-hepatitis drug candidates for the development of drugs for hepatitis.

Those skilled in the art can devise many modifications and other embodiments within the scope and spirit of the presently disclosed inventions. Indeed, variations in the materials, methods, drawings, experiments examples and embodiments described may be made by skilled artisans without changing the fundamental aspects of the disclosed inventions. Any of the disclosed embodiments can be used in combination with any other disclosed embodiment.

The disclosed embodiments, examples and experiments are not intended to limit the scope of the disclosure nor to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

What is claimed is:

1. A method of culturing human organ cells, comprising:
a) culturing the human organ cells in a non-human plasma culture medium;
b) replacing the non-human plasma culture medium with human plasma medium; and
c) culturing the human organ cells in human plasma medium wherein the human plasma medium is refreshed with new human plasma medium at least once every, one (1) to fifteen (15) days or longer.

2. The method of claim 1, wherein the human organ cells are cultured in a non-human plasma culture medium for at least 20 minutes.

3. The method of claim 1, wherein the human organ cells in human plasma medium are cultured for at least 15 days.

4. The method of claim 1, wherein the human organ cells in human plasma medium are cultured for up to about 15 days.

5. The method of claim 1, wherein the human organ cells are freshly isolated, previously cryopreserved, or obtained from a continuously cultured cell line.

6. The method of claim 1, wherein the human plasma medium comprises 70 to 100% human plasma.

7. The method of claim 1, wherein the human plasma medium consists of 100% plasma.

8. The method of claim 1, wherein the human organ cells are selected from hepatocytes, renal cells, pulmonary epithelial cells, enterocytes, cardiomyocytes, vascular endothelial cells, skeletal muscle cells, smooth muscle cells, neurons, lymphocytes, red blood cells, keratinocytes, adipocytes, adrenal cells, thyroid cells, thymic cells, connective tissue cells, testicular cells, ovarian cells, or a combination thereof.

9. A method of culturing human hepatocytes, comprising:
a) culturing the human hepatocytes in a non-human plasma culture medium;
b) replacing the non-human plasma culture medium with human plasma medium; and,
c) culturing the human organ cells in human plasma medium wherein the human plasma medium is refreshed with new human plasma medium at least once every, one (1) to fifteen (15) days or longer.

10. The method of claim 9, wherein the human hepatocytes are cultured in a non-human plasma culture medium for at least 20 minutes.

11. The method of claim 9, wherein the human hepatocytes in human plasma medium are cultured for at least 15 days.

12. The method of claim 9, wherein the human hepatocytes in human plasma medium are cultured for up to about 15 days.

13. The method of claim 9, wherein the human hepatocytes are freshly isolated, previously cryopreserved or obtained from a continuously cultured cell line.

14. The method of claim 9, wherein the human plasma medium comprises 70-100% human plasma.

15. The method of claim 9, wherein the human plasma medium consists of 100% plasma.

16. A method of culturing human hepatocytes, comprising:
a) isolating human hepatocytes from a human liver;
b) culturing the human hepatocytes in a non-human plasma culture medium;
c) replacing the non-human plasma culture medium with human plasma medium; and,
d) culturing the human organ cells in human plasma medium wherein the human plasma medium is refreshed with new human plasma medium at least once every, one (1) to fifteen (15) days or longer.

17. The method of claim 16, wherein the human hepatocytes are cultured in a non-human plasma culture medium for at least 20 minutes.

18. The method of claim 16, wherein the human hepatocytes in human plasma medium are cultured for at least 15 days.

19. The method of claim 16, wherein the human hepatocytes in human plasma medium are cultured for up to about 15 days.

20. The method of claim 16, wherein the human plasma medium comprises 70-100% human plasma.

21. The method of claim 19, wherein the human plasma medium consists of 100% plasma.

22. A cell culture system for evaluating test compound properties, liver disease progression ad/or cell biology processes, comprising:
a cell culture vessel comprising one or more assay wells, wherein the assay wells comprise human plasma culture medium and human organ cells.

23. The cell culture system of claim 22, further comprising a test compound.

24. The cell culture system of claim 22, wherein the test compound is a drug, a drug candidate, an industrial chemical, an environmental pollutant, a pesticide, an insecticide, a biological chemical, or a chemical.

25. The cell culture system of claim 22, wherein the test compound properties comprise drug metabolism, drug-drug interactions, and pharmacology of the test compound.

26. The cell culture system of claim 22, wherein liver disease comprises hepatitis A infection, hepatitis B infection or hepatitis C infection.

27. The cell culture system of claim 22, wherein cell biology processes comprise gene expression, protein synthesis or response to hormones.

28. The cell culture system of claim 22, wherein the human cells are primary cells or cell lines.

29. The cell culture system of claim 22, wherein the human organ cells were previously cryopreserved.

30. The cell culture system of claim 22, wherein the human organ cells are selected from hepatocytes, renal cells, pulmonary epithelial cells, enterocytes, cardiomyocytes, vascular endothelial cells, skeletal muscle cells, smooth muscle cells, neurons, lymphocytes, red blood cells, keratinocytes, adipocytes, adrenal cells, thyroid cells, thymic cells, connective tissue cells, testicular cells, ovarian cells, or a combination thereof.

31. The cell culture system of claim 22, wherein the cell culture vessel is a single well plate.

32. The cell culture system of claim 22, wherein the cell culture vessel is a multi-well plate.

33. The cell culture system of claim 22, wherein the human plasma is prepared from blood obtained from human donors.

34. The cell culture system of claim 22, wherein the human plasma is pooled from multiple donors.

35. The cell culture system of claim 22, wherein the human plasma medium comprises 70-100% human plasma.

36. The cell culture system of claim 22, wherein the human plasma medium consists of 100% plasma.

37. A cell culture system for evaluating test compound properties, liver disease progression and/or cell biology processes, comprising:
a cell culture vessel comprising one or more assay wells, wherein the assay wells comprise human plasma culture medium and human hepatocytes.

38. The cell culture system of claim 37, further comprising a test compound or virus.

39. The cell culture system of claim 37, wherein the test compound is a drug, a drug candidate, an industrial chemical, an environmental pollutant, a pesticide, an insecticide, a biological chemical, or a chemical.

40. The cell culture system of claim 37, wherein the test compound properties comprise drug metabolism, drug-drug interactions, and pharmacology of the test compound.

41. The cell culture system of claim 37, wherein liver disease comprises hepatitis A infection, hepatitis B infection or hepatitis C infection.

42. The cell culture system of claim 37, wherein cell biology processes comprise gene expression, protein synthesis or response to hormones.

43. The cell culture system of claim 37, wherein the human hepatocytes are primary cells or cell lines.

44. The cell culture system of claim 37, wherein human hepatocytes were previously cryopreserved.

45. The cell culture system of claim 37, wherein the cell culture vessel is a single well plate.

46. The cell culture system of claim 37, wherein the cell culture vessel is a multi-well plate.

47. The cell culture system of claim 37, wherein the human plasma is prepared from blood obtained from human donors.

48. The cell culture system of claim 37, wherein the human plasma is pooled from multiple donors.

49. The cell culture system of claim 37, wherein the human plasma medium comprises 90-100% human plasma.

50. The cell culture system of claim 37, wherein the human plasma medium consists of 100% plasma.

51. A method for evaluating test compound properties, the method comprising:
a) providing a cell culture system of claims 22-50;
b) introducing the test compound into the assay well;
c) incubating the test compound for 0.5 h to 10 days at 33 to 40° C.; and,
d) performing an end point assay of the human cells or cell culture medium to determine properties of the test compound.

52. The method of claim 51, wherein the end point assay comprises measuring cell viability, cell function, gene expression, protein expression, metabolite formation or metabolite profiles.

53. The method of claim 51, wherein the end point assay comprises measuring disappearance of a test compound and the appearance of metabolites, measuring effect of a first test compound on metabolism of co-administered second test compound, or measuring pharmacological effects on the human organ cells.

54. The method of claim 51, wherein the system is incubated at 37° C. at 5% CO₂.

55. The method of claim 51, wherein the test compound properties comprise drug metabolism, drug-drug interactions, and pharmacology of the test compound.

56. The method of claim 51, wherein the test compound is a drug, a drug candidate, an industrial chemical, an environmental pollutant, a pesticide, an insecticide, a biological chemical, or a chemical.

57. A method for evaluating liver disease progression, the method comprising:
a) providing a cell culture system of claims 22-50;
b) culturing the human hepatocytes in the human plasma culture medium; and,
c) performing an end point assay of the human hepatocytes or cell culture medium for evaluation of cell biology, chemistry and/or molecular biology of the human hepatocytes relevant to the disease progression.

58. The method of claim 57, wherein the liver disease comprises hepatitis A infection, hepatitis B infection or hepatitis C infection.

59. The method of claim 57, further comprising introducing a hepatitis virus into the assay well.

60. The method of claim 59, further comprising introducing a test compound into the assay well.

61. The method of the claim 60, wherein the test compound is a drug, a drug candidate, an industrial chemical, an environmental pollutant, a pesticide, an insecticide, a biological chemical, or a chemical.

62. The method of claim 57, wherein the system is incubated at 37° C. at 5% CO₂.

63. A method for evaluating cell biology processes, the method comprising:
a) providing a cell culture system of claims 22-50;
b) culturing the human organ cells in the human plasma culture medium; and,
c) performing an end point assay of the human organ cells or cell culture medium for evaluation of cell biology, chemistry and molecular biology of the human organ cells relevant to the cell biology processes being investigated.

64. The method of claim 63, further comprising introducing a test compound into the assay well.

65. The method of claim 64, wherein the test compound is a drug, a drug candidate, an industrial chemical, an environmental pollutant, a pesticide, an insecticide, a biological chemical, or a chemical.
